(19) United States
(12) Patent Application Publication
(10) Pub. No.: US 2005/0171073 A1
(43) Pub. Date: Aug. 4, 2005

(54) Composition and method for treatment and chemoprevention of prostate cancer

(76) Inventors: Mitchell S. Steiner, Germantown, TN (US); Sharan Raghow, Collierville, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 11/038,468

(22) Filed: Jan. 21, 2005

Related U.S. Application Data
(63) Continuation-in-part of application No. 10/611,056, filed on Jul. 2, 2003.
Application No. 10/611,056 is a continuation-in-part of application No. 09/707,766, filed on Nov. 8, 2000, now Pat. No. 6,632,447.
Application No. 09/707,766 is a continuation-in-part of application No. 09/531,472, filed on Mar. 20, 2000, now Pat. No. 6,413,533.
Application No. 09/531,472 is a continuation-in-part of application No. 09/436,208, filed on Nov. 8, 1999, now abandoned.
Application No. 09/436,208 is a continuation-in-part of application No. 09/306,958, filed on May 7, 1999, now Pat. No. 6,265,448.
(60) Provisional application No. 60/084,602, filed on May 7, 1998.

(51) Int. Cl. A61K 31/58; A61K 31/138; A61K 31/57

(52) U.S. Cl. 514/171; 514/651

(57) ABSTRACT

This invention relates to compositions and methods of use thereof in the prevention of prostate carcinogenesis in a subject; prevention of the recurrence of, suppression, inhibition or reduction of the incidence of prostate carcinogenesis in a subject; treatment of a subject with prostate cancer; suppression, inhibition or reduction of the incidence of prostate cancer in a subject; treatment of a subject with pre-malignant lesions of prostate cancer; and/or suppression, inhibition or reduction of the incidence of pre-malignant lesions of prostate cancer in a subject.

### CROSS REFERENCE TO RELATED APPLICATION

This application is a Continuation-in-Part application of U.S. Ser. No. 10/611,056, filed Jul. 2, 2003, which is a Continuation-in-Part application of U.S. Ser. No. 09/707,766, filed Nov. 8, 2000, which is a Continuation-in-Part application of U.S. Ser. No. 09/531,472, filed Mar. 20, 2000, now U.S. Pat. No. 6,413,533, which is a Continuation-in-Part application of U.S. Ser. No. 09/436,208, filed Nov. 8, 1999, which is a Continuation-in-Part application of U.S. Ser. No. 09/306,958, filed May 7, 1999, now U.S. Pat. No. 6,265,448, which claims priority of U.S. Provisional Application No. 60/084,602, filed May 7, 1998, which are hereby incorporated by reference in their entirety

### FIELD OF INVENTION

This invention relates to compositions comprising an antiestrogen and an antiandrogen and methods of use thereof. The invention provides compositions for such use in: 1) preventing prostate carcinogenesis in a subject; 2) preventing the recurrence of, suppressing, inhibiting or reducing the incidence of prostate carcinogenesis in a subject; 3) treating a subject with prostate cancer; 4) suppressing, inhibiting or reducing the incidence of prostate cancer in a subject; 5) treating a subject with pre-malignant lesions of prostate cancer; and/or 6) suppressing, inhibiting or reducing the incidence of pre-malignant lesions of prostate cancer in a subject.

### BACKGROUND OF THE INVENTION

Prostate cancer is one of the most frequently occurring cancers among men in the United States, with hundreds of thousands of new cases diagnosed each year. Unfortunately, over sixty percent of newly diagnosed cases of prostate cancer are found to be pathologically advanced, with no cure and a dismal prognosis. One approach to this problem is to find prostate cancer earlier through screening programs and thereby reduce the number of advanced prostate cancer patients Another strategy, however, is to develop drugs to reduce its incidence.

One third of all men over 50 years of age have a latent form of prostate cancer that may be activated into the life-threatening clinical prostate cancer form. The frequency of latent prostatic tumors has been shown to increase substantially with each decade of life from the 50s (5.3-14%) to the 90s (40-80%). The number of people with latent prostate cancer is the same across all cultures, ethnic groups, and races, the frequency of clinically aggressive cancer is markedly different. This suggests that environmental factors and/or genetics play a role in activating latent prostate cancer. Thus, the development of chemoprevention strategies against prostate cancer may have the greatest overall impact both medically and economically against prostate cancer.

Because of the high incidence and mortality of prostate cancer, it is imperative to develop chemoprevention strategies against this devastating disease. Understanding those factors that contribute to prostate carcinogenesis including the initiation, promotion, and progression of prostate cancer will provide molecular mechanistic clues as to appropriate points of intervention to prevent or halt the carcinogenic process. New innovative approaches are urgently needed at both the basic science and clinical levels to decrease the incidence of prostate cancer as well as to halt or cause the regression of latent prostate cancer. As the frequency of prostate cancer escalates dramatically at the same aces when men are confronted by other competing causes of mortality, simply slowing the progression of prostate adenocarcinoma may be both a more suitable and cost effective health strategy.

Further, as prostate intraepithelial neoplasia (PIN) and high-grade prostate intraepithelial neoplasia (HGPIN), is in the direct causal pathway to prostate cancer and its presence specifically portends an increased risk of prostate cancer, men diagnosed with prostate intraepithelial neoplasia have dramatic changes in their quality of life. The only way to diagnose prostate intraepithelial neoplasia (PIN) is by prostate biopsy. Once the diagnosis of prostate intraepithelial neoplasia is made, however, the standard of medical care is that the patient must be subjected to more frequent biopsies and physician visits. In addition, there is great patient and physician anxiety because the diagnosis of prostate cancer is imminent. Currently, there is no treatment available for patients who have prostate intraepithelial neoplasia.

### SUMMARY OF THE INVENTION

In one embodiment, this invention provides a composition comprising an antiandrogen, and a compound represented by the structure of Formula (I): 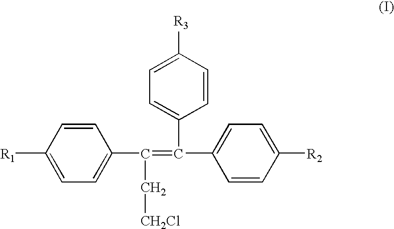
In another embodiment the compounds is an N-oxide, ester, pharmaceutically acceptable salt, hydrate, or any combination thereof, of the compound represented by Formula (I)

In one embodiment, the antiandrogen is a SARM

In one embodiment, SARM is characterized by the structure of the Formula (II): 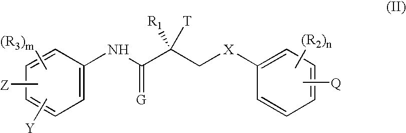
wherein
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 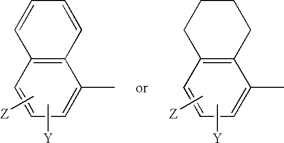
Z is NO₂, CN, COR, COOH, or CONHR;
Y is CF₃, F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

In another embodiment, G in the above the above compound is O. In another embodiment, X in Formula (II) above is O. In another embodiment, T in Formula (II) above is OH. In another embodiment, R₁ in Formula (II) above is CH₃. In another embodiment, Z in Formula (II) above is NO₂. In another embodiment, Z in Formula (II) above is CN. In another embodiment, Y in Formula (II) above is CF₃. In another embodiment, Q in Formula (II) above is NCS. In another embodiment, Q in Formula (II) above is in the para position. In another embodiment, Z in Formula (II) above is in the para position. In another embodiment, Y in Formula (II) above is in the meta position. In another embodiment, G in Formula (II) above is O, T is OH, R₁ is CH₃, X is O, Z is NO₂, Y is CF₃, and Q is NCS.

In another embodiment SARM is characterized by the structure of Formula (III): 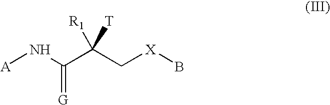
wherein
X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 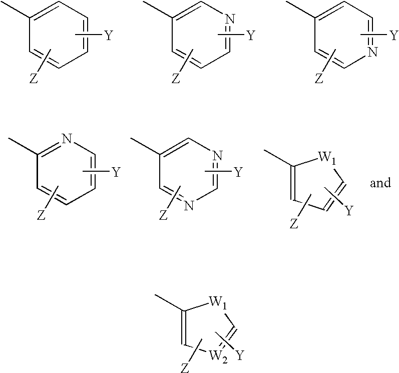
B is a ring selected from: 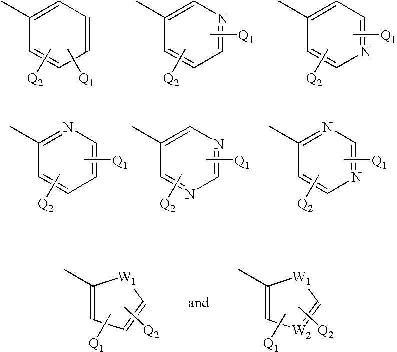
   wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 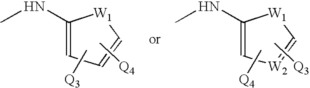
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO and
wherein all unspecified positions can be substituted or unsubstituted.

In one embodiment, G in Formula (III) above is O. In another embodiment, X in Formula (III) above is O. In another embodiment, T in Formula (III) above is OH. In another embodiment, R₁ in Formula (III) above is CH₃. In another embodiment, Z in Formula (III) above is NO₂. In another embodiment, Z in Formula (III) above is CN. In another embodiment, Y in Formula (III) above is CF₃. In another embodiment, Q₁ in Formula (III) above is NCS. In another embodiment, Q₁ in Formula (III) above is in the para position. In another embodiment, Z in Formula (III) above is in the para position. In another embodiment, Y in Formula (III) above is in the meta position. In another embodiment, G in Formula (III) above is O, T is OH, R₁ is CH₃, X is O, Z is NO₂, Y is CF₃, and Q₁ is NCS.

In another embodiment SARM is characterized by the structure Formula (IV): 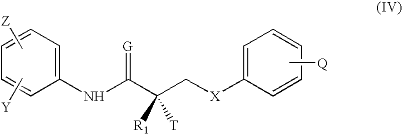
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH;
and R₁ is CH₃, CH₂F, CHF₂, CF₃, CH₂CH₃, or CF₂CF₃

In one embodiment, G in Formula (IV) above is O. In another embodiment, X in Formula (IV) above is O. In another embodiment, T in Formula (IV) above is OH. In another embodiment, R₁ in Formula (IV) above is CH₃. In another embodiment, Z in Formula (IV) above is NO₂. In another embodiment, Z in Formula (IV) above is CN. In another embodiment, Y in Formula (IV) above is CF₃. In another embodiment, Q in Formula (IV) above is NCS. In another embodiment, Q in Formula (IV) above is in the para position In another embodiment, Z in Formula (IV) above is in the para position. In another embodiment, Y in Formula (IV) above is in the meta position In another embodiment, G in Formula (IV) above is O, T is OH, R₁ is CH₃, X is O, Z is NO₂, Y is CF₃, and Q is NCS

In another embodiment, the SARM is characterized by the structure of Formula V, or its analog, isomer, metabolite, derivative, pharmaceutically acceptable salt, N-oxide, hydrate or any combination thereof; 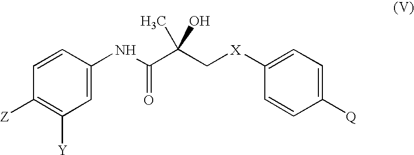
wherein:
X is O, CH₂, NH, Se, PR, or NR;
Z is NO₂, CN, COR, COOH or CONHR;
Y is CF₃, F, Br, Cl, I, CN, or SnR₃;
R is alkyl, a haloalkyl, aryl, phenyl, halo, alkenyl or hydroxyl; and
Q is halogen, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
and a pharmaceutically acceptable carrier or diluent.

In one embodiment, X in compound V is O. In another embodiment, Z in compound V is NO₂. In another embodiment, Z in compound V is CN. In another embodiment, Y in compound V is CF₃. In another embodiment, Q in compound V is NCS

In another embodiment this invention provides a method of suppressing, or in another embodiment, inhibiting or in another embodiment, preventing prostate cancer, the method comprising the step of administering to said subject an antiandrogen and a compound represented by the structure of formula (I), in an amount effective to suppress, or in another embodiment inhibit or in another embodiment prevent prostate cancer in the subject.

In another embodiment this invention provides a method of preventing prostate cancer relapse, the method comprising the step of administering to said subject an antiandrogen and a compound represented by the structure of formula (I), in an amount effective to prevent prostate cancer relapse in the subject.

In one embodiment this invention provides a method of treating prostate cancer in a subject, comprising the step of administering to a subject a composition comprising an antiandrogen and a compound represented by the structure of formula (I), in an amount effective to treat prostate cancer in said subject.

### DETAILED DESCRIPTION OF THE INVENTION

In one embodiment, this invention provides a composition comprising an antiandrogen, and a compound, represented by the structure of formula (I); or its N-oxide, ester, pharmaceutically acceptable salt, hydrate, or any combination thereof 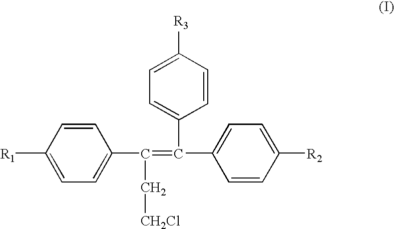
wherein
R₁ and R₂, which can be the same or different, are in one embodiment
H or OH;
R₃ is OCH₂CH₂NR₄R₅,
R₄ and R₅, which can be the same or different, are H or an alkyl group of 1 to about 4 carbon atoms,
a pharmaceutically acceptable carrier, excipient, flow agent, processing aid or diluent.

In one embodiment, the antiandrogen is a selective androgen receptor modulator (SARM). In one embodiment, the SARM binds the receptor. In another embodiment the SARM activates the receptor. The androgen receptor (AR) in one embodiment refers to a ligand-activated transcriptional regulatory protein In one embodiment, following androgen binding, the complex mediates induction of male sexual development.

In one embodiment, the antiandrogen or SARM compounds of the present invention bind reversibly or in another embodiment, irreversibly to the androgen receptor (AR). In one embodiment, the androgen receptor is an androgen receptor of a mammal, such as in another embodiment a human. In one embodiment, reference to reversible binding of a compound to a receptor refer to the ability of a compound to detach from the receptor after binding. Such detaclument may vary as a function of time, in one embodiment, or in another embodiment compound binding affinity or combination thereof.

In one embodiment, the present invention provides a selective androgen receptor modulator (SARM) compound having in-vivo androgenic and anabolic activity following interaction.

In one embodiment, androgenic activity is in non-sex organs. In one embodiment, the SARM of this invention possesses agonist activity peripherally and antagonist activity in a gonad

In one embodiment, the antiandrogen is a SARM, which in one embodiment is characterized by the structure of the following Formula 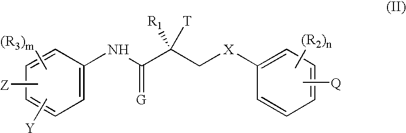
wherein
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 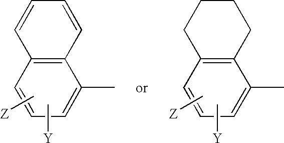
Z is NO₂, CN, COR, COOH, or CONHR;
Y is CF₃. F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted

In one embodiment, G in Formula (II) above is O. In another embodiment, X in Formula (II) above is O. In another embodiment, T in Formula (II) above is OH. In another embodiment, R₁ in Formula (II) above is CH₃. In another embodiment, Z in Formula (II) above is NO₂. In another embodiment, Z in Formula (II) above is CN. In another embodiment, Y in Formula (II) above is CF₃. in another embodiment, Q₁ in Formula (II) above is NCS. In another embodiment, Q₁ in Formula (II) above is in the para position. In another embodiment, Z in Formula (II) above is in the para position In another embodiment, Y in Formula (II) above is in the meta position. In another embodiment, G in Formula (II) above is O, T is OH, R₁ is CH₃, X is O, Z is NO₂, Y is CF₃, and Q₁ is NCS.

In another embodiment the antiandrogen is a selective androgen receptor modulator (SARM) characterized by the following Formula: 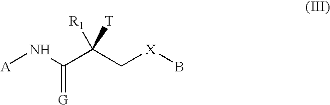
wherein
X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 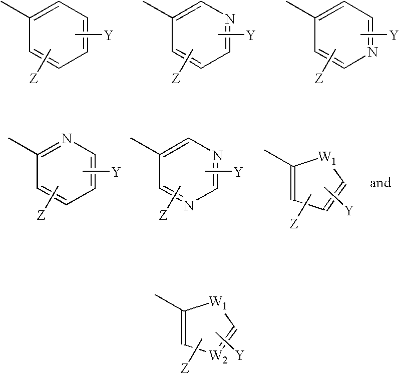
B is a ring selected from: 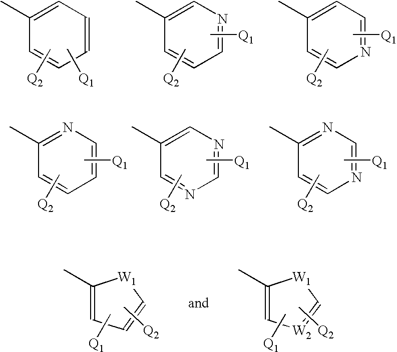
   wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 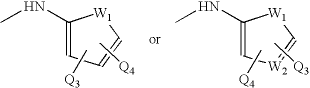
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO and
wherein all unspecified positions can be substituted or unsubstituted.

In one embodiment, G in the above the above compound is O. In another embodiment, X in Formula (III) above is O. In another embodiment, T in Formula (III) above is OH. In another embodiment, R₁ in Formula (III) above is CH₃. In another embodiment, Z in Formula (III) above is NO₂. In another embodiment, Z in Formula (III) above is CN. In another embodiment, Y in Formula (III) above is CF₃. In another embodiment, Q in Formula (III) above is NCS. In another embodiment, Q in Formula (III) above is in the para position. In another embodiment, Z in Formula (III) above is in the para position. In another embodiment, Y in Formula (III) above is in the meta position. In another embodiment, G in Formula (III) above is O, T is OH, R₁ is CH₃, X is O, Z is NO₂, Y is CF₃, and Q is NCS.

In one embodiment the antiandrogen is a SARM, characterized by the structure of the following formula: 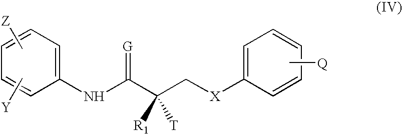
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH;
and R₁ is CH₃, CH₂F, CHF₂, CF₃, CH₂CH₃, or CF₂CF₃

In one embodiment, G in Formula (IV) above is O. In another embodiment, X in Formula (IV) above is O. In another embodiment, T in Formula (IV) above is OH. In another embodiment, R₁ in Formula (IV) above is CH₃. In another embodiment, Z in Formula (IV) above is NO₂. In another embodiment, Z in Formula (IV) above is CN. In another embodiment, Y in Formula (IV) above is CF₃. In another embodiment, Q in Formula (IV) above is NCS. In another embodiment, Q in Formula (IV) above is in the para position. In another embodiment, Z in Formula (IV) above is in the para position. In another embodiment, Y in Formula (IV) above is in the meta position. In another embodiment, G in Formula (IV) above is O, T is OH, R₁ is CH₃, X is O, Z is NO₂, Y is CF₃, and Q is NCS.

In one embodiment the antiandrogen is a selective androgen receptor modulator (SARM) characterized by the structure of formula, V or its analog, isomer, metabolite, derivative, pharmaceutically acceptable salt, N-oxide, hydrate or any combination thereof, 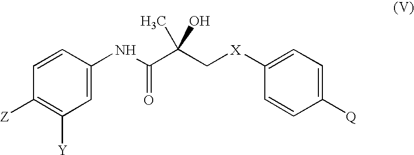
wherein:
X is O, CH₂, NH, Se, PR, or NR;
Z is NO₂, CN, COR, COOH or CONHR;
Y is CF₃, F, Br, Cl, I, CN, or SnR₃;
R is alkyl, a haloalkyl, aryl, phenyl, halo, alkenyl or hydroxyl; and
Q is halogen, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
and a pharmaceutically acceptable carrier or diluent.

In one embodiment, X in compound V is O. In another embodiment, Z in compound V is NO₂. In another embodiment, Z in compound V is CN. In another embodiment, Y in compound V is CF₃. In another embodiment, Q in compound V is NCS

In one embodiment, this invention provides an analog of the compound of in Formula (II-V) above. In another embodiment, this invention provides a derivative of the compound of in Formula (II-V) above. In another embodiment, this invention provides an isomer of the compound of in Formula (II-V) above. In another embodiment, this invention provides a metabolite of the compound of in Formula (II-V) above. In another embodiment, this invention provides a pharmaceutically acceptable salt of the compound of in Formula (II-V) above. In another embodiment, this invention provides a pharmaceutical product of the compound of in Formula (II-V) above. In another embodiment, this invention provides a hydrate of the compound of in Formula (II-V) above. In another embodiment, this invention provides an N-oxide of the compound of in Formula (II-V) above. In another embodiment, this invention provides a combination of any of an analog, derivative, metabolite, isomer, pharmaceutically acceptable salt, pharmaceutical product, hydrate or N-oxide of the compound of in Formula (II-V) above.

In another embodiment, this invention provides a composition comprising an antiandrogen such as: diethylstilbestrol (DES), megestrol, finasteride, dutasteride, epristeride, osaterone, chlormadinone, cyproterone, 4-androsten-3,17-dione, 5-methylsulfonyl[3,2-b]furansteroid, 17-alpha-methyl-testosterone, Flutamide, Nilutamide, Bicalutamide, or a combination thereof.

In one embodiment, the composition comprise optical isomers of the selective androgen receptor modulator (SARM) compound. In another embodiment, the composition comprises optically-active or racemic forms of the selective androgen receptor modulator (SARM). In another embodiment, the composition comprises stereroisomeric form of a selective androgen receptor modulator (SARM), or mixtures thereof In one embodiment, the SARMs are the pure (R)-isomers. In another embodiment, the SARMs are the pure (S)-isomers. In another embodiment, the SARMs are a mixture of the (R) and the (S) isomers In another embodiment, the SARMs are a racemic mixture comprising an equal amount of the (R) and the (S) isomers.

In one embodiment, the optically active forms of SARM may be prepared by recrystallization techniques, which resolves of the racemic form, or in another embodiment, by synthesis from optically-active starting materials, or in another embodiment, by chiral synthesis, or in another embodiment, by chromatographic separation using a chiral stationary phase.

In one embodiment, the invention includes pharmaceutically acceptable salts of the compounds used in compositions and methods of the present invention. In one embodiment, the invention also includes N-oxides of the compounds used in compositions and methods of the present invention. In another embodiment, pharmaceutically acceptable salts are prepared from phenolic compounds by its treatment with an inorganic base, for example, sodium hydroxide. In another embodiment, esters of the phenolic compounds are made with aliphatic and aromatic carboxylic acids, for example, acetic acid and benzoic acid esters.

In another embodiment the selective androgen receptor modulator (SARM) is in a dosage that is in one embodiment between about 0.1 to about 20 mg/Kg. In another embodiment, the selective androgen receptor modulator (SARM) is in a dosage that is between about 0.5 to about 4.0 mg/Kg. The dosage may be in the range of 0.5-20 mg/day. In another embodiment the dosage is in the range of 15-20 mg/day. In another embodiment the dosage is in the range of 10-15 mg/day. In another embodiment the dosage is in a range of 5-10 mg/day. In another embodiment the dosage is in the range of 0.1-5 mg/day. In another embodiment the dosage is in the range of 0.5-4.5 mg/day. In another embodiment the dosage is in the range of 1-5 mg/day. In another embodiment the dosage is in the range of 2-5 mg/day. In another embodiment the dosage is in the range of 3-5 mg/day. In another embodiment the dosage is in the range of 4-5 mg/day. In another embodiment the dosage is in the range of 4.5-5 mg/day. In another embodiment the dosage is in the range of 0.1-0.5 mg/day. In another embodiment the dosage is in the range of 0.6-1 mg/day. In another embodiment the dosage is in the range of 1-1.5 mg/day. In another embodiment the dosage is in the range of 1.5-2 mg/day. In another embodiment the dosage is in the range of 2-2.5 mg/day. In another embodiment the dosage is in the range of 2.75-3 mg/day. In another embodiment the dosage is in the range of 3-3.5 mg/day. In another embodiment the dosage is in the range of 3.5-4 mg/day. In another embodiment the dosage is in the range of 4-4.5 mg/day. In another embodiment the dosage is in the range of 4.5-5 mg/day.

In one embodiment, the present invention provides compositions comprising an antiestrogen in combination with a selective androgen receptor modulator (SARM) which, in another embodiment may be useful in applications in prostate carcinogenesis, as is discussed further hereinbelow.

In one embodiment, this invention provides a composition comprising an antiandrogen and a compound represented by the structure of formula (I). In one embodiment, the antiandrogen is represented by the N-oxide, ester, pharmaceutically acceptable salt, hydrate, or any combination of Formula (I).

In one embodiment the compound of Formula (I), is a selective estrogen receptor modulator (SERM). In another embodiment, the invention provides compositions, which comprise another SERM, such as Tamoxifen, Droloxifene, Idoxifene, Clomiphene, Enclomiphene, Zuclomiphene, LY 353381, EM 800 (SCH 57050) or its metabolite EM 652, Lasofoxifene (CP 336,156) or Levormeloxifene, or a combination thereof. In another embodiment the SERM may be an analog, derivative, isomer, metabolite, pharmaceutically acceptable salt, ester, or N-oxide, or a mixture of the abovementioned compounds.

A “Selective Estrogen Receptor Modulator” (SERM) refers to a compound that modulates or affects ER activity, expression or combination thereof In one embodiment, the SERM exhibits activity as an agonist or antagonist of an estrogen receptor (e.g., ER.alpha. or ER.beta.) in a tissue-dependent manner. In one embodiment the SERM compounds of this may act as estrogen receptor agonists in some tissues and as antagonists in other tissue types. In one embodiment agonist activity is in an organ, which is not a gonad

In one embodiment, the SERM of formula I is Toremifene.

In one embodiment, the invention encompasses the use of analogs, derivatives, isomers, metabolites, pharmaceutically acceptable salts, esters, or N-oxides, or any mixtures thereof of the SERM compounds described herein.

In one embodiment, the SERM is an isomer, which may encompass an embodiment as described herein.

In one embodiment, the daily dosage of the compound represented by the structure of Formula I may be varied over a wide range from 0.01 to 1000 mg per adult human per day. In another embodiment, the dosages ranges from 0.1 to 200 mg/day. For oral administration in one embodiment, the compositions are provided in the form of tablets containing 0.01 to 1000 mg, particularly 0.01, 0.05, 0.1, 0.5, 1.0, 1.5, 2, 2.5, 3.0, 5.0, 6.0, 10.0, 15.0, 25.0, 50.0, 75, 100, 125, 150, 175, 180, 200, 225, and 500 milligrams of the compound represented by the structure of Formula I for the symptomatic adjustment of the dosage to the patient to be treated.

In one embodiment, the pharmaceutical composition comprises about 20 mg to about 60 mg of the antiestrogen. In another embodiment, the pharmaceutical composition comprises about 20 mg of the antiestrogen. In another embodiment, the pharmaceutical composition comprises about 40 mg of the antiestrogen. In another embodiment, the pharmaceutical composition comprises about 60 mg of the antiestrogen. In another embodiment, the pharmaceutical composition comprises about 20 mg to about 30 mg of the antiestrogen. In another embodiment, the pharmaceutical composition comprises about 30 mg to about 40 mg of the antiestrogen. In another embodiment, the pharmaceutical composition comprises about 40 mg to about 50 mg of the antiestrogen. In another embodiment, the pharmaceutical composition comprises about 50 mg to about 60 mg of the antiestrogen. In another embodiment, the pharmaceutical composition comprises about 60 mg to about 70 mg of the antiestrogen. In another embodiment, the pharmaceutical composition comprises about 70 mg to about 80 mg of the antiestrogen. In one embodiment the antiestrogen is the compound of formula I.

The dosage of the compound of formula (I) may be in the range of 5-80 mg/day. In another embodiment the dosage is in the range of 35-66 mg/day. In another embodiment the dosage is in the range of 40-60 mg/day. In another embodiment the dosage is in a range of 45-60 mg/day. In another embodiment the dosage is in the range of 15-25 mg/day. In another embodiment the dosage is in the range of 55-65 mg/day. In another embodiment the dosage is in the range of 45-60 mg/day. In another embodiment the dosage is in the range of 60-80 mg/day. In another embodiment the dosage is 20 mg/day. In another embodiment the dosage is 40 mg/day. In another embodiment the dosage is 60 mg/day. In another embodiment the dosage is 80 mg/day.

In one embodiment, the dosage is 20 mg/day. In another embodiment, the dosage is 40 mg/day. In another embodiment, the dosage is 60 mg/day. In one embodiment, the dosage is 20 mg/day and the antiestrogen is toremifene. In another embodiment, the dosage is 40 mg/day and the antiestrogen is toremifene. In another embodiment, the dosage is 60 mg/day and the antiestrogen is toremifene. In another embodiment, the dosage is 80 mg/day and the antiestrogen is Toremifene.

In one embodiment the composition comprises an analog, derivative, isomer, metabolite, or any combination thereof, of a selective estrogen receptor modulator (SERM) . In another embodiment, the composition comprises an antiestrogen. In one embodiment, the antiestrogen is a non DNA adduct forming antiestrogen, its analog, derivative, isomer, metabolite, or combination thereof.

The antiestrogen agents, for example, the compounds of formula (I) can be prepared according to procedures described in the previously cited U.S. Pat. No. 4,696,949 and 5,491,173 to Toivola et al., the contents of which are incorporated by reference in their entirety herein.

Embodiments of antiestrogens in compositions of, and for use in the methods of this invention include: toremifene analogs, or synthetics thereof; selective estrogen receptor modulators (SERMS), triphenylethylenes, such as for example; droloxifen, idoxifene, tamoxifen, (2)-4-OH-tamoxifen; arzoxifene; chromans such as levomeloxifene and centchroman; benzothiophenes such as raloxifene and LY 353381; naplithalens such as CP336156; phytoestrogens such as isoflavonoids including daidzein, genistein, yenoestrogens; coumestrol: zearalenone; daidzein; apigenin; waempferol; phioretin; biochanin A; naringenin; formononetin; ipriflavone; quercetin; chrysin; flavonoids; flavones, isoflavones, flavanones, and chalcones); coumestans; mycoestrogens; resorcydic acid factone: nafoxideneand equol, and lignan including enterodiol and enterolactone; ICI 164,384, ICI 182, 780; TAT-59, EM-652 (SCG 57068), EM-800 (SCH57050), EM-139, EM-651, EM-776, peptide antagonists of human estrogen receptors, dihydronapthalene , benzothiopheneor, or analog, derivative, isomer, metabolite or a combination thereof

In one embodiment, the present invention provides a composition comprising a) from about 20 mg to about 80 mg of a compound represented by the structure of formula (I) and/or an analog or metabolite thereof, its N-oxide, ester, pharmaceutically acceptable salt, hydrate, or any combination and b) from about 10 to about 100 mg of another SERM compound represented by the structure of formula (IV) and/or an analog or metabolite thereof, its N-oxide, ester, pharmaceutically acceptable salt, hydrate or any combination thereof.

In one embodiment, the term “about” refers to a deviation from the range of 1-20%, or in another embodiment, of 1-10%, or in another embodiment of 1-5%, or in another embodiment, of 5-10%, or in another embodiment, of 10-20%.

In one embodiment, the compositions of the invention may further comprise a GnRH agonist, a GnRH antagonist, an anticancer drug, a 5-alpha reductase inhibitor, an aromatase inhibitor or a progestin, or combination thereof.

In one embodiment, the term gonadotropin-releasing hormone (GnRH), is used interchangeably with luteinizing hormone-releasing hormone (“LHRH”) and refers to decapeptide (pGlu-His-Trp-Ser-Tyr-Gly-Leu-Arg-Pro-Gly—NH₂). In another embodiment, GnRH agonist, or in another embodiment antagonist affect GnRH release from the hypothalamus, the pituitary gland stimulation, biosynthesis release of luteinizing hormone (LH), or follicle-stimulating hormone (FSH). In one embodiment, administration of GnRH agonist depletes gonadotropin and down-regulates the receptor, suppressing steroidal hormone production as a function of time, or in another embodiment, dosage, or in another embodiment, combination thereof. In another embodiment, GnRH agonist, or in another embodiment, antagonist are administered to suppress gonadal sex-steroid production.

In one embodiment, the GnRH has an amino acid sequence of pGlu-His-Trp-Ser-Tyr-(D)Ser[0′bu]-Arg-Pro-EtNH-Ac (SEQ ID NO.1)

In one embodiment, the pharmaceutical composition comprises about 0.01 mg to about 2 mg of the GnRH. In another embodiment, the pharmaceutical composition comprises about 0.01 mg of the GnRH. In another embodiment, the pharmaceutical composition comprises about 0.1 mg of the GnRH. In another embodiment, the pharmaceutical composition comprises about 0.5 mg of the GnRH. In another embodiment, the pharmaceutical composition comprises about 0.01 mg to about 1 mg of the GnRH. In another embodiment, the pharmaceutical composition comprises about 1.0 mg to about 1.5 mg of the GnRH. In another embodiment, the pharmaceutical composition comprises about 1.5 mg to about 1.75 mg of the GnRH. In another embodiment, the pharmaceutical composition comprises about 1.75 mg to about 2.0 mg of the GnRH. In another embodiment, the pharmaceutical composition comprises about 0.8 mg to about 1.0 mg of the GnRH. In another embodiment, the pharmaceutical composition comprises about 0.9 mg of the GnRH.

In one embodiment, a composition of this invention further comprises a 5 alpha reductase inhibitor (5-ARI). In one embodiment, the 5 alpha reductase inhibitor is MK-906, a product of Merck, Sharp & Dohme (Mc Connell et al., J. Urol. 141: 239A, 1989). In another embodiment, the 5 alpha reductase inhibitor is 17β-N,N-diethylcarbamoyl-4-methyl-4-aza-5.alpha.-androstan-3-one (4-MA) (Brooks et al., Endocrinology 109: 830, 1981; Liang et al., Endocrinology 112: 1460, 1983). In another embodiment, the 5 alpha reductase inhibitor is a 4-azasteroid, which can be formed as in Liang et al., J. Biol. Chem. 259: 734-739, 1984; and in Brooks et al., Steroids 47: 1-19, 1986.). In another embodiment, the 5 alpha reductase inhibitor is a 6-methylene-4-pregnene-3,20-dione, for example, as described (Petrow et al., J. Endocrinol. 95: 311-313, 1982). In another embodiment, the 5 alpha reductase inhibitor is a 4-methyl-3-oxo-4-aza-5.alpha.-pregnane-3 0(s) carboxylate (Kadohama et al., J. Natl. Cancer Inst 74: 475-486, 1985).

The enzyme 5.alpha-reductase catalyzes the conversion of testosterone to dihydrotestosterone (DHT), and an inhibitor of this enzyme in one embodiment, prevents, or in another embodiment inhibits the conversion such that in one embodiment, it reduces DHT levels without significantly affecting testosterone levels.

In one embodiment, 5-ARI, administered in combination with antiestrogen and antiandrogen, treats diseases, disorders and conditions which are stimulated, exacerbated, or prolonged by elevated androgen production, or in another embodiment, accompanied by elevated estrogen production

In one embodiment, the present invention further comprises a 5-alpha-reductase inhibitor and/or in another embodiment, metabolite thereof, its N-oxide, ester, pharmaceutically acceptable salt or hydrate thereof.

In one embodiment, the pharmaceutical composition comprises about 1 mg to about 50 mg of the 5-ARI. In another embodiment, the pharmaceutical composition comprises about 1 mg of the 5-ARI. In another embodiment, the pharmaceutical composition comprises about 5 mg of the 5-ARI. In another embodiment, the pharmaceutical composition comprises about 2.5 mg of the 5-ARI. In another embodiment, the pharmaceutical composition comprises about 1 mg to about 10 mg of the 5-ARI. In another embodiment, the pharmaceutical composition comprises about 10 mg to about 25 mg of the 5-ARI. In another embodiment, the pharmaceutical composition comprises about 25 mg to about 40 mg of the 5-ARI. In another embodiment, the pharmaceutical composition comprises about 40 mg to about 50 mg of the 5-ARI. In another embodiment, the pharmaceutical composition comprises about 3 mg to about 5 mg of the 5-ARI. In another embodiment, the pharmaceutical composition comprises about 4.5 mg of the 5-ARI.

In one embodiment, dosage of the antiandrogen which in another embodiment is represented by the structure of formulas (II-V), the antiestrogens, which in another embodiment is represented by the structure of Formula (I), may be further reduced by up to 75% when the compositions further comprise SARM, SERM, GnRH agonist, a GnRH antagonist, a 5-alpha reductase inhibitor (5-ARI), an anticancer drug, an aromatase inhibitor, a progestin or combination thereof

In another embodiment, the invention provides a composition comprising an antiestrogen, which, in another embodiment is represented by the structure of Formula (I), in a dosage between 5 to about 100 mg/Kg, an antiandrogen, which in another embodiment is represented by the structure of Formulas (II-V) in a dosage between 0.01-5.0 mg/Kg and from about 1 to about 50 mg/Kg of a 5-alpha-reductase inhibitor compound and/or in another embodiment, metabolite thereof, its N-oxide, ester, pharmaceutically acceptable salt or hydrate and combinations thereof.

In another embodiment, the invention provides a composition comprising an antiestrogen, which, in another embodiment is represented by the structure of Formula (I), in a dosage between 5 to about 100 mg/Kg, an antiandrogen, which in another embodiment is represented by the structure of Formulas (II-V) in a dosage between 0.01-5 mg/Kg and from about about 0.1 to about 5 mg/Kg of a GnRH compound, which in another embodiment is a LHRH represented by SEQ ID NO.1 and/or in another embodiment, an ester, chelates, pharmaceutically acceptable salt, hydrate or combination thereof.

In one embodiment, the compositions may further comprise cytokines, or in another embodiment growth factors In one embodiment the cytokines are IFN, alpha or beta or in another embodiment interleukin (IL) 1, IL-2, IL-4, IL-6, IL-7, IL-12, or in another embodiment tumor necrosis factor (TNF) α, TNF-β, or in another embodiment granulocyte colony stimulating factor (G-CSF), granulocyte/macrophage CSF (GM-CSF).

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In one embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdennally, intramuscularly, intravenously, intradermally, subcutaneously, intravaginally, intraperitonealy, intraventricularly, intracranially or intratumorally.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-15 33 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral. In one embodiment, the pharmaceutical composition is administered parenterally, paracancerally, transmucosally, transdermally, intrarnuscularly, intravenously, intradermally, subcutaneously, intraperitonealy, intraventricularly, intracranially, or intratumorally.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticies of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

This invention also provides in one embodiment, methods of use of the compositions mentioned hereinabove.

In another embodiment the invention provides compositions for use in preventing prostate carcinogenesis in a subject, and in another embodiment preventing the recurrence of prostate carcinogenesis in a subject. In one embodiment, the present invention provides use of a composition of this invention in suppressing prostate carcinogenesis in a subject, or in another embodiment, in inhibiting prostate carcinogenesis in a subject, or in another embodiment in reducing the incidence of prostate carcinogenesis in a subject.

In one embodiment, the term “therapeutically effective amount” refers to that amount which provides a therapeutic effect for a given condition and administration regimen.

In one embodiment, the compositions of the present invention improve the therapeutic efficiency of cancer therapy, including in one embodiment very early stage cancer patients with visible tumor mass who, in one embodiment may may, or in another embodiment may not be candidates for surgery. In another embodiment, late stage cancer patients with larger tumors or metastases may be treated by the methods and compositions of the present invention. In another embodiment, the compositions of this invention are administered prior to surgery, or in another embodiment following surgery, as adjunct therapy.

In one embodiment it will be desirable to deliver the compositions disclosed herein parenterally, intravenously, intramuscularly, or even intraperitoneally. Such approaches are well known to the skilled artisan, some of which are further described, for example, in U.S. Pat. No. 5,543,158; U.S. Pat No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. It must be stable under the conditions of manufacture and storage and must be preserved against the contaminating action of microorganisms, such as bacteria and fungi.

In another embodiment, it will be preferable to include isotonic agents, for example, sugars or sodium chloride. In other embodiments, prolonged absorption of the injectable compositions will be desireable. Prolonged absorption of the injectable compositions can be brought about by the use of agents delaying absorption, for example, aluminum monostearate and gelatin, in the compositions

Parenteral vehicles include in certain embodiments sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

For topical administration to body surfaces using, for example, creams, gels, drops, and the like, the antiestrogen and the selective androgen receptor modulator (SARM) agents or their physiologically tolerated derivatives such as salts, esters, N-oxides, and the like can be prepared and applied as solutions in one embodiment, or suspensions in another embodiment, or emulsions in another embodiment or foams in another embodiment or gels in another embodiment or creams in another embodiment or ointments in another embodiment, in a physiologically acceptable diluent with a pharmaceutical carrier in one embodiment, or without a pharmaceutical carrier in another embodiment.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

For use in medicine, the salts of the selective androgen receptor modulator (SARM) may be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid

In another embodiment this invention provides a method of suppressing, or in another embodiment, inhibiting or in another embodiment, preventing prostate cancer, the method comprising the step of administering to said subject an antiandrogen and a compound represented by the structure of formula (I), in an amount effective to suppress, or in another embodiment inhibit or in another embodiment prevent prostate cancer in the subject.

In another embodiment this invention provides a method of preventing prostate cancer relapse, the method comprising the step of administering to said subject an antiandrogen and a compound represented by the structure of formula (I), in an amount effective to prevent prostate cancer relapse in the subject.

In one embodiment this invention provides a method of treating prostate cancer in a subject, comprising the step of administering to a subject a composition comprising an antiandrogen and a compound represented by the structure of formula (I), in an amount effective to treat prostate cancer in said subject.

The present invention provides compositions and methods for prevention and/or inhibition of Prostate Cancer (PC), including all stages of PC. In one embodiment, PC may refer to advanced prostate cancer, or in another embodiment, to locally advanced prostate cancer, which in one embodiment has extended through the prostate capsule. PC may include in another embodiment stage C disease under the American Urological Association (AUA) system, in another embodiment, stage C1-C2 disease under the Whitmore-Jewett system, and in another embodiment, stage T3-T4 and N+ disease under the TNM (tumor, node, metastasis) system.

In one embodiment, the compositions and methods of this invention promote, an anti-tumor response, and is at least 10-50% above the basal (i.e., untreated) level. In another embodiment, an effective dose is an amount of a compound that, when administered as described herein, is capable of preventing, inhibiting or suppressin the treated disease stage, as described herein.

In one embodiment the methods are affected via administration of a composition of this invention In another embodiment, the methods are affected via administration of the antiestrogens and antiandrogen individually set as combined therapy. In one embodiment, composition of the present invention is administered as a combination preparation for simultaneous administration to a mammal, preferably a human subject, within a period of time which is enough for the active compounds both antiandrogens, including in one embodiment selective androgen receptor modulators (SARM) and Toremifene and other antiestrogens to prevent prostate carcinogenesis in a subject, and/or in another embodiment, to prevent the recurrence of prostate carcinogenesis in a subject.

The present invention provides, in one embodiment, for the use of a compound of Formula (I) and/or an antiestrogen and an antiandrogen, including a selective androgen receptor modulator (SARM), each provided independently, or in combination within a single composition, for the uses claimed herein. In one embodiment, such use may include administration of the compounds at different fixed amounts, or in another embodiment at the same amounts or at another embodiment, the amounts of each or in another embodiment the amounts of both components may vary as a function of time.

In one embodiment, this invention provides for the simultaneous, or in another embodiment, sequential administration, which in one embodiment, refers to administration at successive points in time, wherein each increment is equal, or in another embodiment, varies as a function of time, or symptoms of the subject, or in another embodiment, staging of the cancer, and others.

Routes and frequency of administration of the compositions, or compositions for use in the methods of the invention described, as well as dosage, will vary from individual to individual, and may be readily established using standard techniques. In one embodiment, administration may be via a route such as, for example, by injection (e.g. intracutaneous, intramuscular, intravenous or subcutaneous), or in another embodiment administration may be intranasal (e.g., by aspiration), or in one embodiment administration may be oral, or in another embodiment, administration may be by catheterization to the affected area. Alternate protocols may be appropriate for individual subjects.

According to an aspect of the invention, and in one embodiment, the subject suffers from prostate cancer and in another embodiment, has been exposed to androgen-deprivation therapy.

In one embodiment, the subject for the methods of this invention, may have an elevated risk of prostate cancer. In another embodiment, the subject has benign prostatic hyperplasia (BPH), prostatic intraepithelial neoplasia (PIN), high-grade prostatic intraepithelial neoplasia (HGPIN), or an abnormally high level of circulating prostate specific antibody (PSA).

In one embodiment, the term “suppressing” refers to the prevention of any stage in the pathogenesis of prostate carcinogenesis. In another embodiment, suppressing refers to the diminishing of precancerous precursor expression, in another embodiment, suppressing refers to reduction in tumor size, in another embodiment, suppressing refers to the extension of the latency period between pre malignant lesions and carcinogenesis, in another embodiment, suppressing refers to the prevention of metastasis of cancer cells into adjacent tissues, in another embodiment, suppressing refers to the reduction of cellular growth.

In one embodiment, the term “treating” includes preventative treatment, or in another embodiment disorder-remitative treatment, or in another embodiment palliative treatment.

In one embodiment, the term “reducing”, refers to alleviating typical disease condition symptoms, or in another embodiment lowering the size of tumors, or in another embodiment, lowering the number the number of tumor foci, or in another embodiment reducing the number of secondary metastasis sites.

In one embodiment, the term “inhibiting” refers to the lessening, or in another embodiment decreasing disease, or in another embodiment increasing the latency period between progression of disease stages as described herein, or in another embodiment affecting partial remission, or in another embodiment, complete remission, or in another embodiment stopping tumor growth.

In one embodiment, the term “progression” refers to increasing in scope, or in another embodiment severity, or in another embodiment advancing, or in another embodiment growing or or in another embodiment becoming worse. In one embodiment. The term “recurrence” refers in one embodiment to the return of a disease after a remission.

In one embodiment, the present invention provides methods of use of the compositions mentioned hereinabove for treating a subject with prostate cancer, or in another embodiment suppressing incidence of prostate cancer in a subject

In one embodiment, the present invention provides methods and compositions for inhibiting the incidence of prostate cancer in a subject or, in another embodiment reducing the incidence of prostate cancer in a subject.

In one embodiment, the present invention provides methods and compositions for treating a subject with pre-malignant lesions of prostate cancer in one embodiment, or suppressing the incidence of pre-malignant lesions of prostate cancer, or in another embodiment, inhibiting the incidence of pre-malignant lesions of prostate cancer in a subject or in another embodiment, reducing the incidence of pre-malignant lesions of prostate cancer in a subject.

In one embodiment, the present invention provides methods and compositions for suppressing or in another embodiment for inhibiting prostate cancer, or in another embodiment latent prostate cancer, or in another embodiment early stage prostate cancer, or in another embodiment locally advanced prostate cancer and is particularly useful for treating subjects having an elevated risk of developing prostate cancer, for example those having in one embodiment benign prostatic hyperplasia (BPH), or prostate intraepithelial neoplasia (PrN) in another embodiment, or high grade prostate intraepithelial neoplasia (HGPIN) in another embodiment, or in another embodiment, an abnormally high level of circulating prostate specific antibody (PSA) or in another embodiment, having a family history of prostate cancer.

Prostate intraepithelial neoplasia (PIN) refers in one embodiment to a precancerous lesion, or precursor of prostatic adenocarcinoma. Prostate intraepithelial neoplasia manifests in one embodiment as abnormal proliferation within the prostatic ducts of premalignant foci of cellular dysplasia and carcinoma in situ without stromal invasion. In one embodiment, effects on PIN represent an endpoint in prostate chemoprevention trials, or in another embodiment, predictive value as a marker for adenocarcinoma.

In another embodiment, the present invention provides a method of suppressing, or in one embodiment inhibiting or in another embodiment reducing the incidence of prostate cancer in a subject, comprising the step of administering to the subject a pharmaceutical composition comprising from about 10 mg to about 80 mg of a compound represented by the structure of formula (I) and/or an analog or metabolite thereof, its N-oxide, ester, pharmaceutically acceptable salt, hydrate, or any combination thereof and antiandrogen that in another embodiment includes SARM, in dosage as described herein.

In one embodiment, the term “administering” refers to bringing a subject in contact with a compound or composition of the present invention. Administration can be accomplished, in one embodiment in vitro, i.e. in a test tube, or in another embodiment in vivo, i.e. in cells or tissues.

The phrase “in combination” refers in one embodiment, to the simultaneous administration of the compound of Formula (I) and SARM.

In one embodiment, the beneficial effect of the methods and compositions of the invention are pharmakinetic or pharmacodynamic co-action resulting from the combination of therapeutic agents. Wherein simultaneous may refer to administration of base within a composition, administration of 2 or more compositions via the same route of administration, or in another embodiment, via different route. In another embodiment, administration is with a single capsule having a fixed ratio of each therapeutic agent or in another embodiment multiple, or in another embodiment single capsules for each of the therapeutic agents.

In one embodiment sequential or in another embodiment substantially simultaneous administration of each therapeutic agent can be effected in one embodiment, by any appropriate route including, but not limited to, oral routes, intravenous routes, intramuscular routes, and direct absorption through mucous membrane, cutaneous or subcutaneous tissues. In another embodiment, the therapeutic agents according to the invention can be administered by the same route or by different routes. In one embodiment, a first therapeutic agent of the combination selected may be administered by intravenous injection while the other therapeutic agents of the combination may be administered orally. Alternatively, in another embodiment, all therapeutic agents may be administered orally or all therapeutic agents may be administered by intravenous injection in another embodiment.

The sequence in which the therapeutic agents are administered is not narrowly critical In one embodiment “in combination” can also embrace the administration of the therapeutic agents as described above in further combination with other biologically active ingredients (such as, but not limited to, an antineoplastic agent) or in another embodiment, non-drug therapies (such as, but not limited to, surgery in one embodiment or radiation treatment in another embodiment).

Where in one embodiment, the combination further comprises radiation treatment, the radiation treatment may be conducted at any suitable time so long as a beneficial effect from the co-action of the combination of the therapeutic agents and radiation treatment is achieved. Therefore, in appropriate cases, the beneficial effect in another embodiment is still achieved when the radiation treatment is temporally removed from the administration of the therapeutic agents, perhaps by days or even weeks.

In one embodiment, the present invention provides methods for hormone therapy, for treating prostate cancer, for delaying the progression of prostate cancer, and for preventing and/or treating the recurrence of prostate cancer, which comprise administering the compound of Formula (I) and/or antiestrogen, and an antiandrogen including SARM as described hereinabove. In another embodiment, treatment may also comprise the administration of GnRH agonists, GnRH antagonists, reversible antiandrogens, antiestrogens, anticancer drugs, 5-alpha reductase inhibitors, aromatase inhibitors, progestins, agents acting through other nuclear hormone receptors, selective estrogen receptor modulators (SERM), progesterone, estrogen, PDE5 inhibitors, apomorphine, bisphosphonate, and one or more additional SARMS.

In one embodiment, the methods of the present invention comprise administering an antiestrogen compound, in combination with an antiandrogen and a GnRH analog. In another embodiment, the methods of the present invention comprise administering a antiandrogen, in combination with a reversible antiandrogen. In another embodiment, the methods of the present invention comprise administering antiandrogen, in combination with an antiestrogen. In another embodiment, the methods of the present invention comprise administering antiestrogen, in combination with an anticancer drug. In another embodiment, the methods of the present invention comprise administering antiestrogen, in combination with a 5-alpha reductase inhibitor. In another embodiment, the methods of the present invention comprise administering antiestrogen, in combination with an aromatase inhibitor In another embodiment, the methods of the present invention comprise administering antiestrogen, in combination with a progestin. In another embodiment, the methods of the present invention comprise administering antiestrogen, in combination with an agent acting through other nuclear hormone receptors. In another embodiment, the methods of the present invention comprise administering antiestrogen, in combination with a selective estrogen receptor modulators (SERM). In another embodiment, the methods of the present invention comprise administering antiestrogen, in combination with a progesterone. In another embodiment, the methods of the present invention comprise administering antiestrogen, in combination with an estrogen. In another embodiment, the methods of the present invention comprise administering antiestrogen, in combination with a PDE5 inhibitor. In another embodiment, the methods of the present invention comprise administering antiestrogen, in combination with apomorphine. In another embodiment, the methods of the present invention comprise administering antiestrogen, in combination with a bisphosphonate. In another embodiment, the methods of the present invention comprise administering antiestrogen, in combination with one or more additional SARMS.

Furthermore, treatment with the antiestrogen compounds of the present invention may precede or follow a DNA-damaging agent treatment by intervals ranging from minutes to weeks. Protocols and methods are known to those skilled in the art. DNA-damaging agents or factors are known to those skilled in the art and refer to any chemical compound or treatment method that induces DNA damage when applied to a cell. Such agents and factors include radiation and waves that induce DNA damage, such as gamma-irradiation, X-rays, UV-irradiation, microwaves, electronic emissions, and the like. A variety of chemical compounds, also described as “chemotherapeutic agents”, function to induce DNA damage, all of which are intended to be of use in the combined treatment methods disclosed herein. Chemotherapeutic agents contemplated to be of use include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA-damaging agents, whether radiation-based or actual compounds, such as the use of X-rays with cisplatin or the use of cisplatin with etoposide.

In another embodiment, the methods may further comprise irradiating a localized tumor site with DNA-damaging radiation such as X-rays, UV-light, gamma-rays, or even microwaves. Alternatively, the tumor cells may be contacted with the DNA-damaging agent by administering to the subject a therapeutically effective amount of a pharmaceutical composition comprising a DNA-damaging compound, such as adriamycin, 5-fluorouracil, etoposide, camptothecin, actinomycin-D, mitomycin C, or more preferably, cisplatin. Agents that damage DNA also include compounds that interfere with DNA replication, mitosis, and chromosomal segregation. Such chemotherapeutic compounds include adriamycin, also known as doxorubicin, etoposide, verapamil, podophyllotoxin, and the like.

Other factors that cause DNA damage and have been used extensively include what are commonly known as gamma-rays, X-rays, and/or the directed delivery of radioisotopes to tumor cells. Other forms of DNA-damaging factors are also contemplated, such as microwaves and UV-irradiation. It is most likely that all of these factors affect a broad range of damage to DNA, on the precursors of DNA, the replication and repair of DNA, and the assembly and maintenance of chromosomes.

In one embodiment, these compounds produce effectively a treatment repeatedly or continuously for a prolonged period of time, which in one embodiment gonadectomy is induced as a result of a desensitizing effect exerted on the hypophysis by a pronounced reduction of release of gonadotropin and steroids. In another embodiment, gonadectomy is a useful therapeutic intervention in the treatment of steroid-dependent gonadal diseases or entities. In one embodiment, gonadectomy is reversible. Synthetic-GnRH antagonists are derivatives competitively inhibiting the native hormone.

As can be readily appreciated by one of ordinary skill in the art, the methods, compositions and pharmaceutical compositions of the present invention are suitable for administration to a manual, preferably a human subject.

It is to be understood that the compositions of this invention include any and every embodiment thereof, may be used in the methods of this invention.

The following examples are presented in order to more fully illustrate the preferred embodiments of the invention. They should in no way be construed, however, as limiting the broad scope of the invention.

### EXAMPLES

### Example I

### Composition Comprising Toremifene and Antiandrogen

The active ingredients Toremifene (Formula I) and Cyproterone (Formula II) are blended together and the following excipients are blended together with the actives: lactose monohydrate, amorphous lactose fast-flo® 316, Avicel® PH102 (mcc), magnesium stearate (lubricant) and colloidal silicon dioxide (flow agent). The blended active and inactive ingredients are filled into white opaque hard gelatin capsules (size one). An embodiment of the concentrations used is listed in Table 1.

TABLE 1
An embodiment of a formulation of a Toremifene/selective androgen
receptor modulator (SARM) Composition

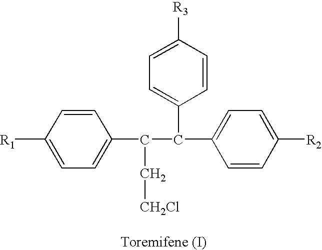

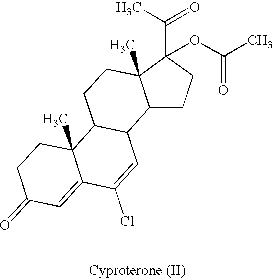

20 mg Toremifene FORMULATION

| | | | Weight/Count | | | |
|---|---|---|---|---|---|---|
| | | Excipient | Per dosage | | Weight/Count | |
| Ingredient: | Manufacturer: | Purpose: | unit: | | Per Batch*: | |
| Toremifene | Shanghai FINC | Active | 20.00 | mg | 10.0 | g |
| | Chem. Technology | | | | | |
| | Co. LTD | | | | | |
| | Laboratories | | | | | |
| Cyproterone | Vinchem, Active | Active | 50 | mg | 25.0 | g |
| Acetate | Pharma | | | | | |
| | Ingredients | | | | | |
| Lactose | Foremost | Diluent/Filler | 80.00 | mg | 40.000 | g |
| Monohydrate, | | | | | | |
| NF | | | | | | |
| (#310 Regular) | | | | | | |
| Lactose | Foremost | Filler/Flow-Aid | 196.45 | mg | 98.225 | g |
| Monohydrate, | | | | | | |
| NF | | | | | | |
| (#316 Fast-Flo | | | | | | |
| Modified, | | | | | | |
| Spray-Dried) | | | | | | |
| Microcrystalline | FMC | Filler/Disintegrant | 30.00 | mg | 15.000 | g |
| Cellulose, NF | | | | | | |
| (Avicel PH102) | | | | | | |
| Silicon Dioxide, | Cabot | Flow-Aid | 1.00 | mg | 0.500 | g |
| Colloidal, | | | | | | |
| USP/NF | | | | | | |
| (Cab-O-Sil M- | | | | | | |
| 5P) | | | | | | |
| Magnesium | Mallinckrodt | Lubricant | 1.55 | mg | 0.775 | g |
| Stearate, NF | | | | | | |
| HyQual | | | | | | |
| Capsule, Hard | Capsugel | Capsule | 1 | (Count) | 500 | (Count) |
| Gelatin | | | | | | |
| Size 1, White | | | | | | |
| Opaque | | | | | | |

*Batch size based on 500 capsules but may change depending on requirements

### Example 2

### Composition Comprising Toremifene and SARM

The active ingredients are Toremifene (Formula I) and the compound represented by Formula III. Excipients are lactose monohydrate, amorphous lactose fast-flo® 316, Avicel® PH102 (mcc), magnesium stearate (lubricant) and colloidal silicon dioxide (flow agent). The blended active and inactive ingredients are filled into white opaque hard gelatin capsules (size one).

TABLE 2
An embodiment of a formulation of a Toremifene/SARM
Composition

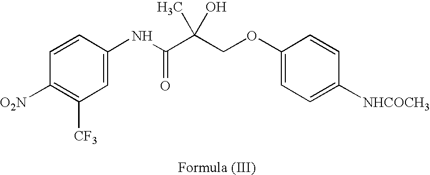

40 mg Toremifene FORMULATION

| | | | Weight/Count | | | |
|---|---|---|---|---|---|---|
| | | Excipient | Per dosage | | Weight/Count | |
| Ingredient: | Manufacturer: | Purpose: | unit: | | Per Batch*: | |
| Toremifene | Shanahai FINC | Active | 40.00 | mg | 10.0 | g |
| | Chem. Technology | | | | | |
| | Co. LTD | | | | | |
| Formula III | ChemSyn | Active | 1.00 | mg | 0.50 | g |
| | Laboratories | | | | | |
| Lactose | Foremost | DiluentiFiller | 80.00 | mg | 40.000 | g |
| Monohydrate, NF | | | | | | |
| (#310 Regular) | | | | | | |
| Lactose | Foremost | Filler/Flow-Aid | 196.45 | mg | 98.225 | g |
| Monohydrate, NF | | | | | | |
| (#316 Fast-Flo | | | | | | |
| Modified, Spray- | | | | | | |
| Dried) | | | | | | |
| Microcrystalline | FMC | Filler/Disintegrant | 30 00 | mg | 15.000 | g |
| Cellulose, NF | | | | | | |
| (Avicel PH102) | | | | | | |
| Silicon Dioxide, | Cabot | Flow-Aid | 1.00 | mg | 0.500 | g |
| Colloidal, | | | | | | |
| USP/NF | | | | | | |
| (Cab-O-Sil M-5P) | | | | | | |
| Magnesium | Mallinckrodt | Lubricant | 1.55 | mg | 0.775 | g |
| Stearate, NF | | | | | | |
| HyQual | | | | | | |
| Capsule, Hard | Capsugel | Capsule | 1 | (Count) | 500 | (Count) |
| Gelatin | | | | | | |
| Size 1, White | | | | | | |
| Opaque | | | | | | |

*Batch size based on 500 capsules but may change depending on requirements

1. A composition comprising an antiandrogen and a compound represented by the structure of formula (I), its N-oxide, ester, pharmaceutically acceptable salt, hydrate, or any combination thereof: 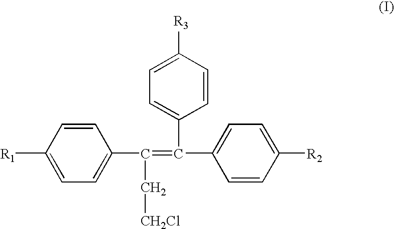
wherein R₁ and R₂, which can be the same or different, are H or OH; R₃ is OCH₂CH₂NR₄R₅, wherein R₄ and R₅, which can be the same or different, are H or an alkyl group of 1 to about 4 carbon atoms, an antiandrogen and another therapeutic agent, a pharmaceutically acceptable carrier, excipient, flow agent, processing aid or diluent.

2. The composition of claim 1, wherein said compound of formula I is Toremifene.

3. The composition of claim 1, wherein said compound of formula I is in a dosage of between 20 to about 80 mg/Kg

4. The composition of claim 1, wherein said antiandrogen is diethylstilbestrol (DES), megestrol, finasteride, dutasteride, epristeride, osaterone, chlormadinone, cyproterone, 4-androsten-3,17-dione, 5-methylsulfonyl[3,2-b]furansteroid, 17-alpha-methyl-testosterone, Flutamide, Nilutamide or Bicalutamide

5. The composition of claim 1, wherein said antiandrogen is a selective androgen receptor modulator (SARM).

6. The composition of claim 5, wherein said selective androgen receptor modulator (SARM) is in a dosage of between about 0.5 to about 4 mg/Kg.

7. The composition of claim 5, wherein the selective androgen receptor modulator (SARM) is represented by the structure of the following formula: 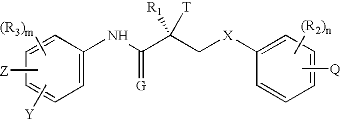
wherein
X is a bond, O, CH₂, NH, S, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 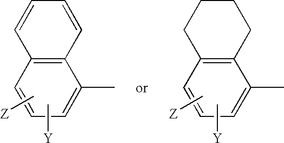
Z is NO₂, CN, COR, COOH, or CONHR;
Y is CF₃ F, Br, Cl, I, CN, or SnR₃;
Q is SCN, NCS, OCN, or NCO;
n is an integer of 1-4;
m is an integer of 1-3; and
wherein all unspecified positions can be substituted or unsubstituted.

8. The composition of claim 5, wherein the selective androgen receptor modulator (SARM) is represented by the structure of the following formula: 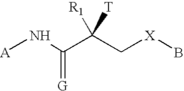
wherein
X is a bond, O, CH₂, NH, S, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 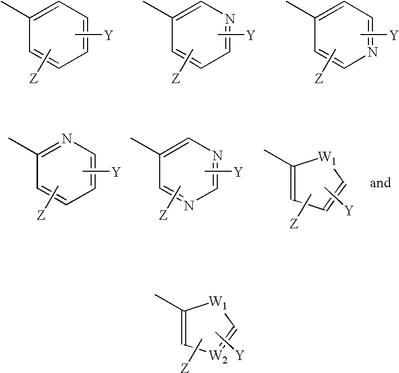
B is a ring selected from: 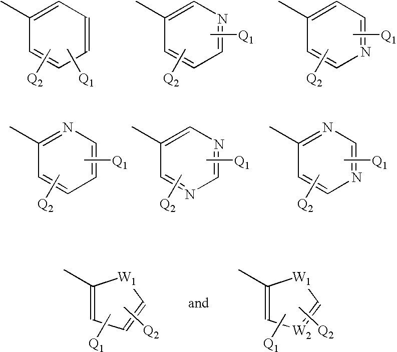
wherein
A and B cannot simultaneously be a benzene ring;
Z is NO₂, CN, COOH, COR, NHCOR or CONHR;
Y is CF₃, F, I, Br, Cl, CN CR₃ or SnR₃;
Q₁ is NCS, SCN, NCO or OCN;
Q₂ is a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 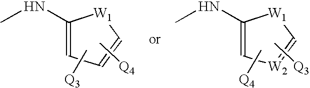
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S;
W₂ is N or NO and
wherein all unspecified positions can be substituted or unsubstituted.

9. The composition of claim 5, wherein the selective androgen receptor modulator (SARM) is represented by the structure of the following formula: 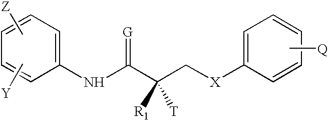
wherein
X is a bond, O, CH₂, NH, S, Se, PR, NO or NR;
G is O or S;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is SCN, NCS, OCN, or NCO;
R is alkyl, haloalkyl, dihaloalkyl, trihaloalkyl, CH₂F, CHF₂, CF₃, CF₂CF₃, aryl, phenyl, halogen, alkenyl or OH;
and R₁ is CH₃, CH₂F, CHF₂, CF₃, CH₂CH₃, or CF₂CF₃

10. The composition of claim 5, wherein said selective androgen receptor modulator (SARM) is a compound represented by the structure of formula, III or its analog, isomer, metabolite, derivative, pharmaceutically acceptable salt, N-oxide, hydrate or any combination thereof; 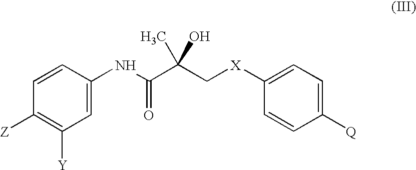
wherein:
X is O, CH₂, NH, Se, PR, or NR;
Z is NO₂, CN, COR, COOH or CONHR;
Y is CF₃, F, Br, Cl, I, CN, or SnR₃;
R is alkyl, a haloalkyl, aryl, phenyl, halo, alkenyl or hydroxyl; and
Q is halogen, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
and a pharmaceutically acceptable carrier or diluent.

11. The composition of claim 1, further comprising an antiestrogen.

12. The composition of claim 11, wherein said antiestrogen is a SERM.

13. The composition of claim 12, wherein said SERM is Tamoxifen, Droloxifene, Idoxifene, Clomiphene, Enclomiphene, Zuclomiphene, LY 353381, EM 800 (SCH 57050) or its metabolite EM 652, Lasofoxifene (CP 336,156), Levormeloxifene or an analog, derivative, isomer, metabolite, pharmaceutically acceptable salt, ester, or N-oxide thereof, or a mixture thereof.

14. The composition of claim 1, wherein said composition further comprises an GnRH agonist, an GnRH antagonist, an anticancer drug, a 5-alpha reductase inhibitor, an aromatase inhibitor, a progestin.

15. The composition of claim 1, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

16. The composition of claim 1, further comprising a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

17. The composition of claim 1, wherein said composition is in the form of a pellet, a tablet, a capsule, a solution, a suspension, a dispersion, an emulsion, an elixir, a gel, an ointment, a cream, or a suppository.

18. The composition of claim 1, wherein said composition is in a form suitable for oral, intravenous, intraaorterial, intramuscular, subcutaneous, parenteral, transmucosal, transdermal, or topical administration.

19. The composition of claim 1, wherein said composition is a controlled release composition.

20. The composition of claim I, wherein said composition is an immediate release composition.

21. The composition of claim 1, wherein said composition is a liquid dosage form.

22. The composition of claim 1, wherein said composition is a solid dosage form.

23. A method of suppressing, inhibiting or preventing prostate cancer, or its relapse, in a subject, comprising the step of administering to said subject the composition of claim I in an amount effective to suppress, inhibit or prevent prostate cancer, or its relapse in said subject.

24. A method of treating prostate cancer in a subject, comprising the step of administering to said subject the composition of claim 1, in an amount effective to treat prostate cancer in said subject.

25. The method as in claim 23, wherein said subject has a premalignant lesion.

26. The method as in claim 25, wherein said premalignant lesion is prostate intraepithelial neoplasia (PIN) or high grade prostate intraepithelial neoplasia (HGPIN).

27. The method as in claim 23, wherein the subject has benign prostate hyperplasia (BPH).

28. A method of treating benign prostate hyperplasia (BPH) in a subject, comprising the step of administering to said subject the composition of of claim 1, in an amount effective to treat benign prostate hyperplasia (BPH) in said subject.

29. A method of treating prostate intraepithelial neoplasia (PIN) in a subject, comprising the step of administering to said subject the composition of of any one of claim 1, in an amount effective to treat prostate intraepithelial neoplasia (PIN) in said subject.

30. A method of treating pre malignant lesions of prostate cancer in a subject, comprising the step of administering to said subject the composition of of any one of claim 1, in an amount effective to treat pre malignant lesions of prostate cancer in said subject.

31. A method of suppressing, inhibiting or reducing the incidence of pre malignant lesions of prostate cancer in a subject, comprising the step of administering to said subject the composition of of any one of claim 1, in an amount effective to suppress, inhibit or reduce the incidence of pre malignant lesions of prostate cancer in said subject.


SEQUENCE LISTING (1 sequence)

SEQ ID NO: 1 - protein, 9 aa, Mammal
  misc_feature (1)..(1): Xaa can be any naturally occurring amino acid
XHWSYSRPX